(19) United States
(12) Patent Application Publication
(10) Pub. No.: US 2007/0017007 A1
(43) Pub. Date: Jan. 25, 2007

(54) Device for covering the eyes

(76) Inventor: Paul McBride, Cornwall (GB)

Correspondence Address: John Wiley Horton;Pennington, Moore, Wilkinson, Bell & Dunbar, 215 S. Monroe St., Tallahassee, FL, 32301, US

(21) Appl. No.: 10/578,404

(22) PCT Filed: Aug. 11, 2004
(86) PCT No.: PCT/GB04/03451

§ 371 Date: Aug. 28, 2006

(30) Foreign Application Priority Data
Aug. 11, 2003 (GB) 0318768.9
Nov. 3, 2003 (GB) 0325589.0

(51) Int. Cl. A61F 9/02

(52) U.S. Cl. 2/426

(57) ABSTRACT

A device for covering the eyes comprises a face-covering element, formed by a first layer of transparent elastic material, which, in use, is stretched to sealingly cover the wearer's eyes, and arms which, in use, extend around the wearer's head to hold the device in place. The device is particularly suitable for use as a swimming mask.

### FIELD OF INVENTION

This invention relates to a device for covering the eyes particularly, though not exclusively, for use in watersports such as swimming.

### BACKGROUND

Swimming is a popular form of recreation throughout the world, many people daily going swimming to keep fit, train for or participate in competition, or simply to have fun. The equipment needed to conduct this sport is inexpensive in contrast to some other sports.

Swimming goggles and masks are used by many swimmers to provide clear vision for the wearer and to help prevent water contacting the eyes, which may be affected by water additives such as chlorine. Due to the awkward shape of the face, the design of such equipment has been constrained to the well-known traditional swimming goggles, which provide two small individual lenses to fit around each eye socket and diving masks, which provide a single visor encompassing the whole region of both eyes. Each design has its own advantages but also limitations.

Diving masks improve visibility and provide a watertight seal but are cumbersome and produce too much drag for use whilst swimming at speed. “Seal Mask” (Aqua Sphere, Vista, Calif., USA) is a more hydrodynamically shaped mask, designed for triathletes who require improved visibility from a mask resistant to being kicked or pulled off during races. However it is still relatively cumbersome compared to the swimming goggles used by speed swimmers. Such goggles are more streamlined but cannot be worn whilst scuba diving because of the pressure generated around the eye socket and reduced visibility compared to a diving mask. In addition, many wearers find that swimming goggles do not provide an adequate seal, allowing leakage into the goggles. As a result of contact with the water, the eyes can become irritated and may also be infected with conditions such as purmra gogglorum, an infection which can result in permanent damage to the eye, including loss of sight.

An example of swimming goggles of the traditional design is described in United States patent U.S. Pat. No. 6,079,054. In this patent the lenses, nose bridge and seals are of unitary construction and are held in position on the wearer's head by an elastic strap.

British patent application GB-A-2326078 relates to swimming goggles mounted directly on a swimming cap. The goggles are held in position on the wearer's face by means of the swimming cap stretching to cover both the wearer's head and goggles. The aim of the construction shown in the application is to reduce the internal misting of goggles experienced by some users. Other integrated swimming cap/goggle designs are disclosed in Japanese patent application IP-A-090140829 and United States patents U.S. Pat. No. 608,539 and U.S. Pat. No. 5,713,078. None of these specifications address the problem of goggle leakage.

European patent application EP-A-1180383 relates to modifications in the design of the traditional swimming goggle in an attempt to improve the seal between the wearer's face and the goggles. The applicant has varied features of the parts of the goggles which make contact with the wearer's face. The invention represents a variation on the design of the traditional goggle style.

### SUMMARY OF THE INVENTION

According to a first aspect of the invention, there is provided a device for covering the eyes comprising a face-covering element, formed by a first layer of transparent elastic material, which, in use, is stretched to sealingly cover the wearer's eyes, and arms which, in use, extend around the wearer's head to hold the device in place. The eyes may be individually sealed by the covering of each eye separately, or both eyes may be covered and sealed together, to prevent external unwanted material from contacting the eyes. The elastic material may be an elastomer material.

It is an advantage of the current invention that the device fits over the eyes of the wearer and encircles the head, making contact with the skin around the forehead, across the nose and cheeks and, in at least some embodiments, at the sides of the face in front of the ears. In a typical application of the device as a swimming mask, this, coupled with the use of elastic material, has the result that water is prevented from contacting a user's eyes, by the generation of an improved seal over that provided by currently available goggles. In addition, the increased pressure around the eye, associated with the use of goggles as the result of the positioning of the goggles in the eye socket, is not a factor in the use of devices in accordance with the invention. This invention represents a completely new approach to solving the problem of designing a device which will cover the wearer's eyes and prevent water making contact with the eyes.

The face-covering element of the device may be of unitary construction.

The thickness of the material forming the face-covering element may vary.

The device, in use, may also cover the wearer's nose, in which case the device may comprise a moulded nose region shaped such that the device can fit around the wearer's nose.

Preferably, the face-covering element may comprise a visor region, which may itself comprise two lens regions, each having an inner and an outer surface. The term “inner surface”, as used throughout this specification, means the surface on the side of the device which, in use, is face-facing. The term “outer surface”, as used throughout this specification, has the opposite meaning. The inner and the outer surface of each lens region may preferably each be part spherical, i.e. having a curvature, in two or more transversely arranged planes, which corresponds with the curvature of part of the surface of a sphere. At least one surface of one lens region may intersect at least one surface of the other lens region. At least one surface of at least one lens region may be treated with an anti-fog coating, for example a polyurethane-based coating such as that obtainable from Hydromer®, New Jersey, USA.

Preferably, the material forming the device may be a plastics material or rubber, preferably polyurethane or a silicone rubber material such as MCP1300T (Mining & Chemical Products Ltd, Wellingborough, UK). In a further alternative, the material forming the visor region may be silicone rubber and the material forming the remainder of the device may be a plastics material (such as polyurethane) or rubber. The visor region may be constructed from a hardened plastics material (e.g. polycarbonate) or glass.

The device may further comprise means for affixing a transparent member, preferably a rigid transparent member, to the device to cover at least part of the face-covering element. Such a member may, for example, provide protection to the eyes from the sun. Alternatively, it may carry a sponsorship logo or add a layer of colour to an otherwise colourless device. Preferably, the transparent member may be affixed to the inner side of the device. The term “inner side”, as used throughout this specification, means the side of the device which, in use, is face-facing. Alternatively, the transparent member may be affixed to the outer side of the device. The transparent member may be removeably or permanently affixed to the device. The transparent member may be not colourless.

In an alternative embodiment of the first aspect of the invention, the device may further comprise a second layer, fixed to the first layer and defining an aperture such that, in use, the wearer's eyes are not covered by the second layer, i.e. so that the user's vision is not impeded. The second layer may preferably be formed from an elastic material. The second layer may be non-transparent and may be a breathable material. The first and second layers may be fixed together by adhesive.

According to a second aspect of the invention there is provided a device for covering the eyes comprising a face-covering element, formed by a first layer of elastic material, which, in use, is stretched to sealingly cover the wearer's eyes, and arms which, in use, extend around the wearer's head to hold the device in place, the device further comprising:

a. an aperture defined by the first layer such that, in use, the wearer's eyes are not covered by the first layer; and

b. a transparent second layer which is fixed to the first layer such that the second layer covers the aperture defined by the first layer.

The second layer may comprise a visor region, itself comprising two lens regions, each having an inner and an outer surface. The inner and the outer surface of each lens region may preferably be part spherical, i.e. having a curvature, in two or more transversely arranged planes, which corresponds with the curvature of part of the surface of a sphere. At least one surface of one lens region may intersect at least one surface of the other lens region. At least one surface of at least one lens region may be treated with an anti-fog coating.

The first layer of the device according to the second aspect of the invention may be non-transparent. The first layer of elastic material may be a breathable material. The first and second layers may preferably be fixed together with adhesive.

A device according to the first or second aspects of the invention may form a band which, in use, encircles a wearer's head. Alternatively, the arms may be releasably fastened together by fastening means. The fastening means may comprise a hook and loop fastening material such as Velcro , a clip device, or a buckle device.

The device may have an upper edge which, in use, makes sealing contact with a wearer's forehead and preferably, in use, may make sealing contact with a wearer's head above the ears. Most preferably the device will make sealing contact with both of these parts of a wearer's head.

The device may have a lower edge which, in use, makes sealing contact with a wearer's face across the nose and preferably has a lower edge which, in use, makes sealing contact with a wearer's face across the cheeks and/or with a wearer's head under the ears. Most preferably the lower edge of the device will make sealing contact with all of these parts of a wearer's head.

The material of the device, in use, may make sealing contact with the side of a wearer's face in front of the ears.

Most preferably, the material of the device will make contact with all of the above-mentioned parts of a wearer's head.

The arms of the device may be of unitary construction with the face-covering element and form cut-outs such that, in use, the wearer's ears are not covered by the device. Alternatively, the arms may be constructed from a different material to the face-covering element and define an aperture in the device such that, in use, the wearer's ears are not covered. The arms may be constructed from a Lycra®-containing material. Preferably, the arms may be arranged such that at least one arm extends around a wearer's head above each ear and/or at least one arm extends around the head below each ear.

According to a third aspect of the invention there is provided a device for covering the eyes comprising a visor region, the visor region comprising two lens regions, each having an inner and an outer surface. The inner and the outer surface of each lens region may preferably be part spherical, i.e. having a curvature, in two or more transversely arranged planes, which corresponds with the curvature of part of the surface of a sphere. At least one surface of one lens region may intersect at least one surface of the other lens region. The material forming the visor region may be a plastics material, glass, or a combination thereof, for example a hardened glass or a laminate polycarbonate/glass bulletproof material. The plastics material may be polyurethane or polycarbonate. Alternatively, the material forming the visor region may be rubber or silicone rubber.

Such a visor region may be incorporated into any device intended to be used to cover the eyes, for example (but not restricted to) sunglasses, a swimming mask or a gas respirator mask.

The device according to the first, second or third aspects of the invention may comprise at least one eye-sealing element. The or each of the eye-sealing element(s) may be of unitary construction with the face-covering element. There may be one eye-sealing element comprising a protrusion from the inner surface of the device, which defines an eye-covering region. There may alternatively be two eye-sealing elements, each comprising a protrusion from the inner surface of the device, each of which defines an eye-covering region. The term “inner surface”, as used throughout this specification, means the surface of the device which, in use, is face-facing: The protrusion(s) may be substantially L-shaped. The or each protrusion(s), in use, may make sealing contact with the wearer's face around the eyes and may space the material forming the inner surface of the or each eye-covering region(s) from the eye or eyes. The or each of the eye sealing element(s) may make contact with the wearer's face around, but not in, each eye socket, advantageously creating a further seal around the or each eye(s) without the creation of undue pressure around each eyeball.

According to a fourth aspect of the invention there is provided a swimming mask in the form of a device according to the invention.

### BRIEF DESCRIPTION OF THE DRAWINGS

Embodiments of the invention will now be described, by way of example only, with reference to the following FIGS. 1 to 24 in which:

FIG. 1 shows an exploded plan view of an embodiment of a device according to the invention;

FIG. 2 shows a front view of the device of FIG. 1;

FIG. 3 shows a side view of the device of FIG. 1;

FIG. 4 shows a front view of an alternative embodiment of a device according to the invention;

FIG. 5 shows a side view of the device of FIG. 4;

FIG. 6 shows a front view of a visor cover for use with the device shown in FIG. 1 or FIG. 4;

FIG. 7 shows a front view of another alternative embodiment of a device according to the invention;

FIG. 8 shows a cross-section of a device according to the invention along the line P-P;

FIG. 9 shows a cross-section along the line P-P of a further alternative embodiment of a device according to the invention;

FIG. 10 shows an elevation of an embodiment of a device according to the invention in position on the wearer;

FIG. 11 shows a side view of an embodiment of a device according to the invention in position on the wearer;

FIG. 12 shows a rear view of an embodiment of a device according to the invention in position on the wearer;

FIG. 13 shows a rear view of an alternative embodiment of a device according to the invention to that shown in FIG. 12, in position on the wearer;

FIG. 14 shows a plan view of a further alternative embodiment of a device according to the invention;

FIGS. 15A and 15B show plan views of component sheets of an alternative embodiment of a device according to the invention;

FIG. 16 shows a cross-section of a device according to the invention along the line Q-Q of FIG. 15B;

FIG. 17 shows an exploded view of an embodiment of a device according to the invention, illustrating the component sheets;

FIG. 18 shows a plan view of an embodiment of a device according to the invention, with a first transparent and second non-transparent sheet fixed together;

FIG. 19 shows a cross-section of a device according to the invention along the line R-R of FIG. 18;

FIG. 20 shows an elevation of a further embodiment of a device according to the invention in position on the wearer;

FIG. 21 shows a side view of a further embodiment of a device according to the invention in position on the wearer;

FIG. 22 shows a rear view of a further embodiment of a device according to the invention in position on the wearer;

FIG. 23 shows a rear view of an alternative embodiment of a device according to the invention to that shown in FIG. 22, in position on the wearer; and

FIG. 24 shows a side view of a further embodiment of a device according to the invention in position on the wearer.

### MODES OF CARRYING OUT THE INVENTION

### EXAMPLE 1

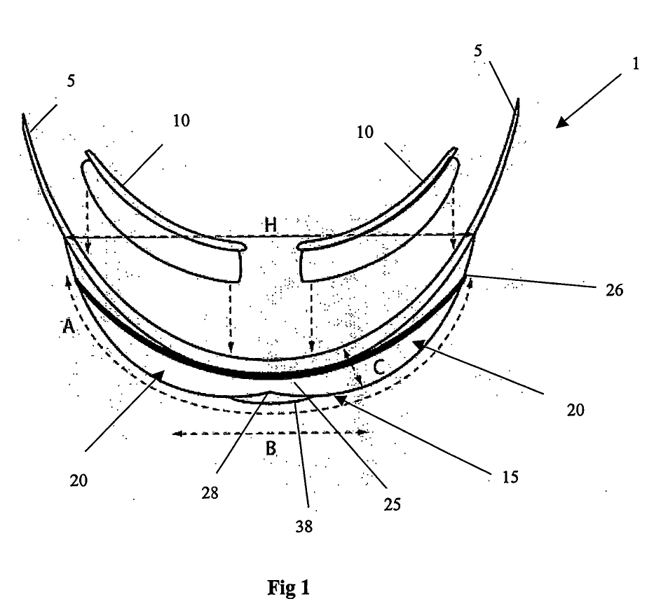

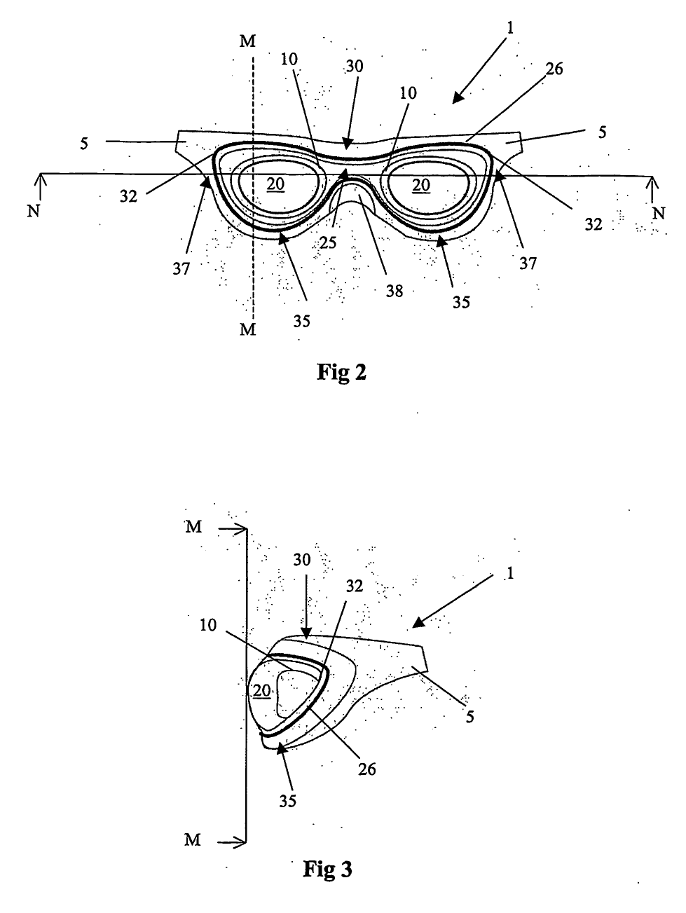

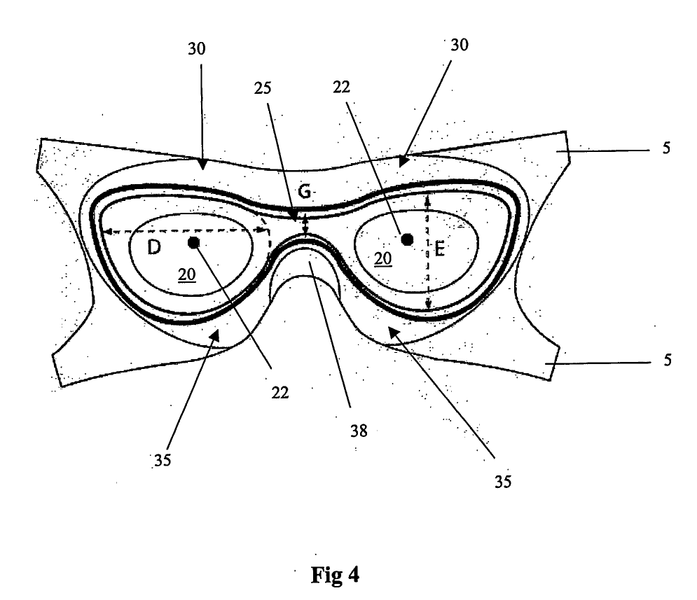

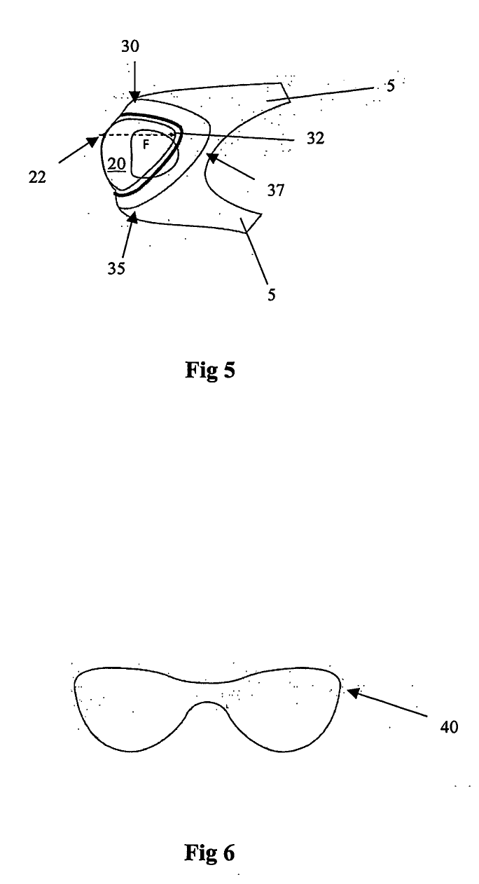

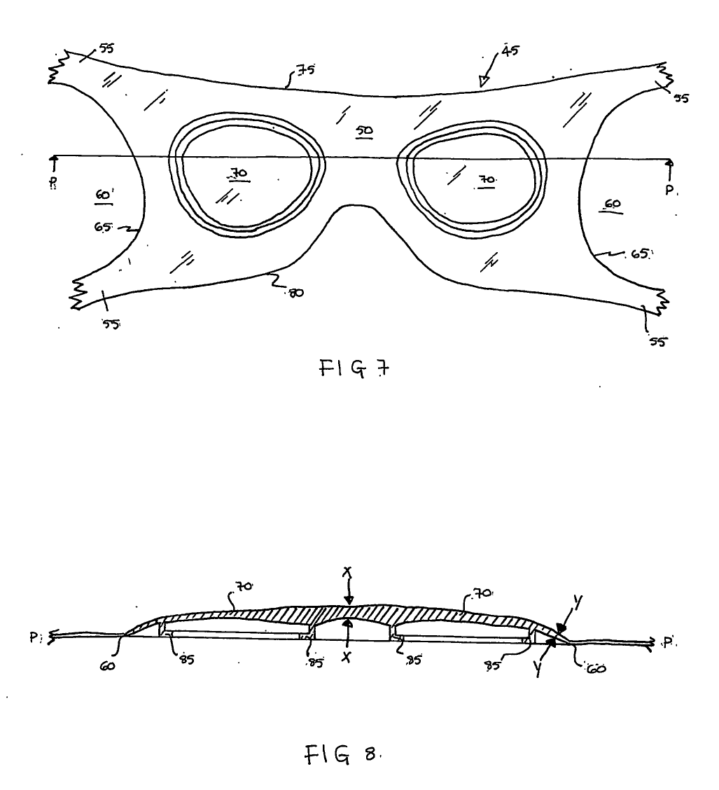

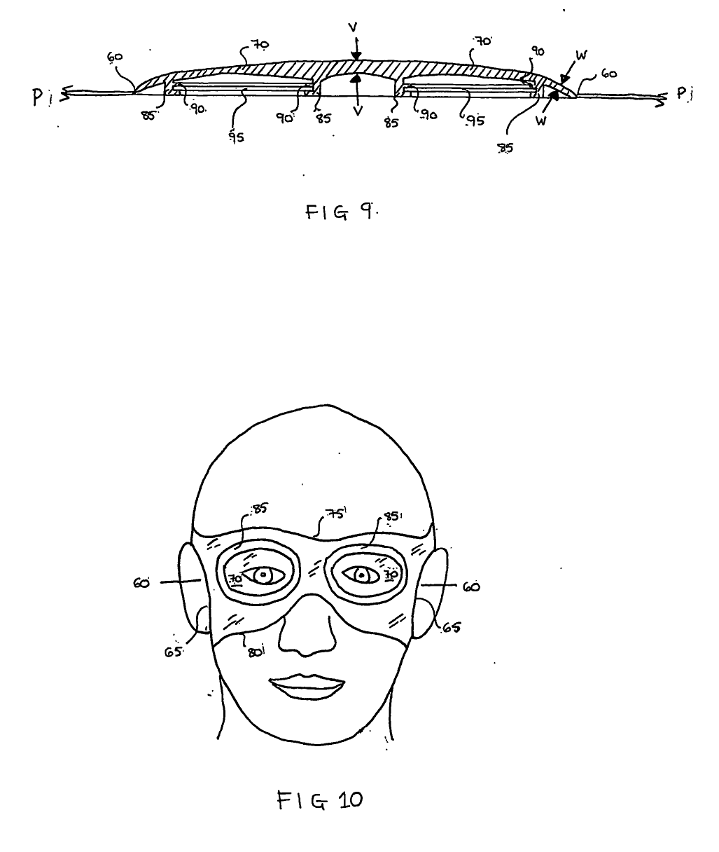

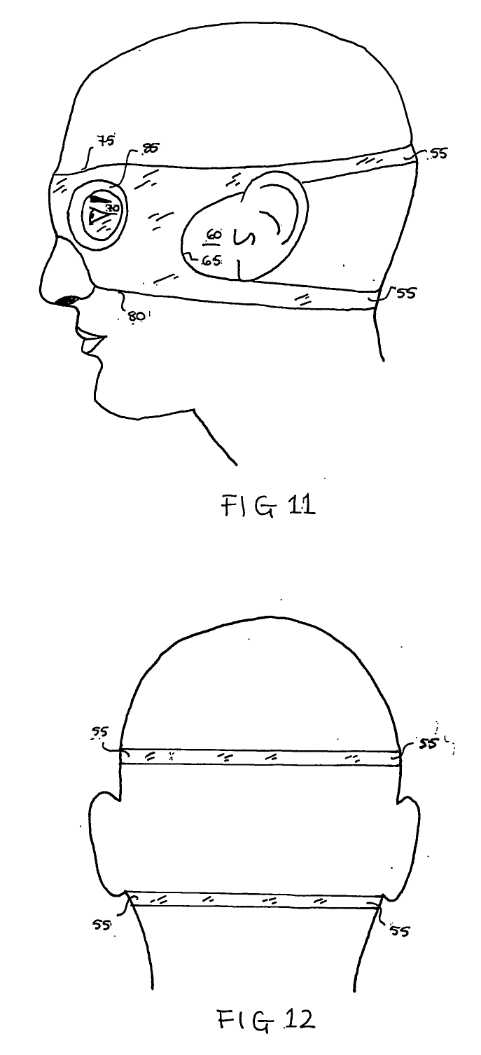

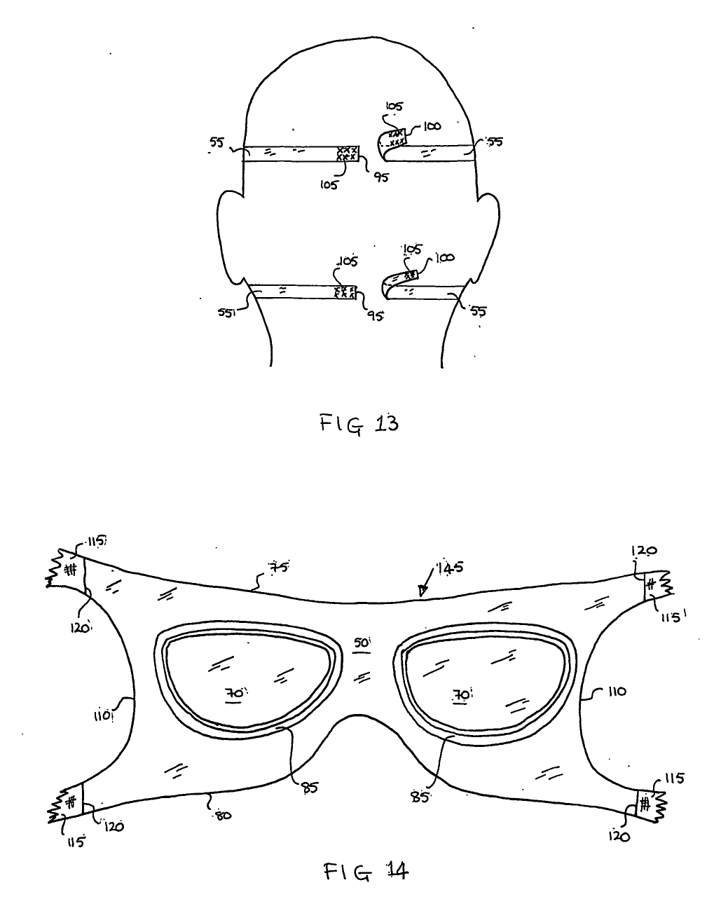

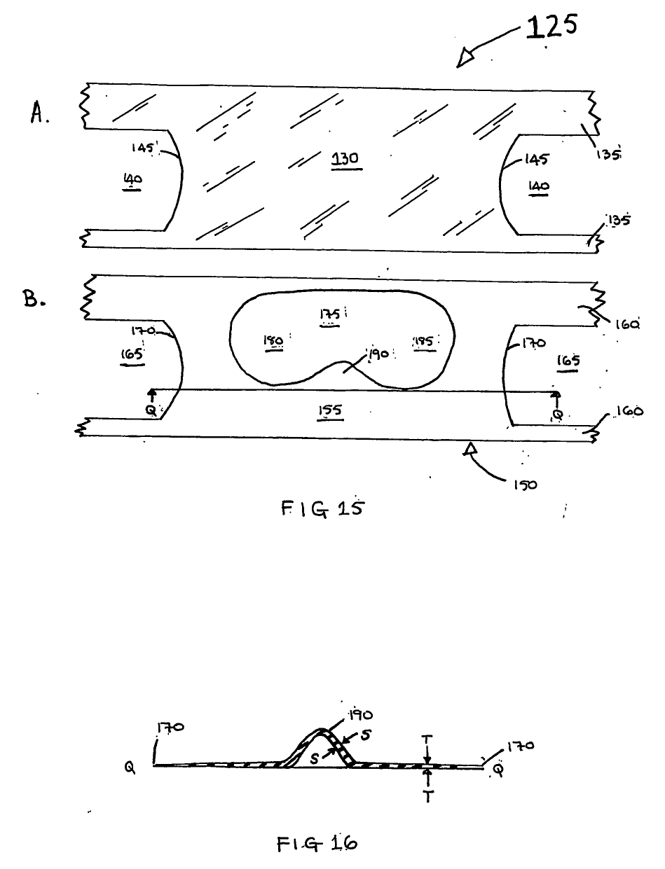

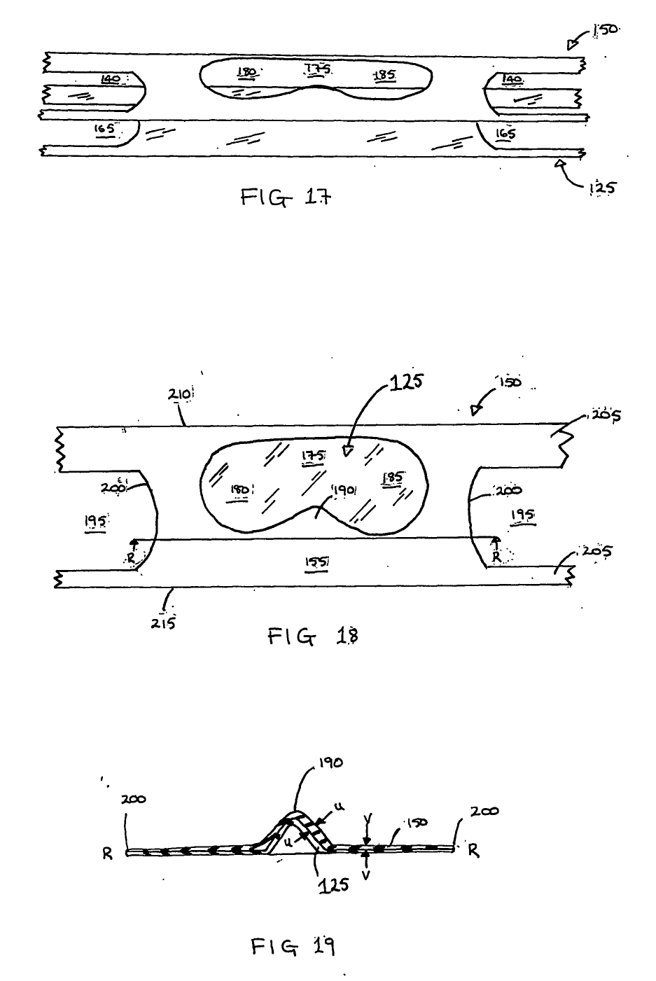

In FIG. 1, the device (1) is a mask which comprises arms (5), eye sealing elements or eye cups (10) and a face-covering element including a visor region, generally indicated at (15). The visor region includes two lens regions (20) and a nose bridge area (25). The mask is of unitary construction, being made of, for example, moulded transparent rubber although, for clarity, the mask is shown in FIG. 1 in an exploded view with the eye cups separated from the body of the mask. A ridge (26), which defines the outline of the visor region (15), is moulded onto the surface of the mask. Advertising and/or sponsorship logos may appear on the material forming the arms (5) or the face-covering element of the device. When the device is in use, each eye sealing element makes contact with the wearer's face around, but not in, the wearer's eye socket.

FIGS. 2 and 3 show a front and side view, respectively, of the mask of FIG. 1. The mask has top (30), bottom (35) and side (37) regions which, in use, make sealing contact with a wearer's face. The eye cups (10) are visible through the transparent lens regions (20). The material of the bottom region (35) abutting the visor nose bridge area (25) is moulded to form a nose contact area (38), to allow the mask, in use, to fit over a wearer's nose. This nose contact area (38) also makes sealing contact with a wearer's face when the device is in use.

As can be seen from the Figures, the visor region includes two lens regions (20). The inner and outer surface of each lens region each has a curvature which is part spherical, i.e. each has a curvature which approximately corresponds with that of part of the surface of a sphere, in at least planes M and N, as shown in FIGS. 2 and 3. The outer surface of one lens region meets the outer surface of the other at a position (28), in the centre of the nose bridge area (25). The height of the visor region is least at the centre of the nose bridge area.

Preferred dimensions of the mask are given, for example only, with reference to FIG. 1. Dotted arrow A indicates the length of the curved material of the visor region as measured between the visor outer extremities (indicated by (32) in FIG. 2), this length being 180mm The linear width between the visor outer extremities (32) is indicated by dotted arrow H and is 45 mm. Dotted arrow B indicates a distance of 80 mm between the “focal points” of each lens region, that is, the points of each lens region where the depth of the mask, shown by dotted arrow C, is greatest (20 mm). These focal points are shown by dots (22) in FIG. 4. FIG. 4 also shows the widths (dotted arrow D, 60 mm) and heights (dotted arrow E, 45 mm) of each lens region of the visor region. The height of the visor region is reduced at the nose bridge area (25) to a height of 15 mm, indicated by arrow G. Dotted arrow F, shown in FIG. 5, indicates the linear distance between the focal point (22) of each lens region and the outer extremities (32) of the visor region to be 30 mm.

The mask can be formed by the well-known process of injection moulding, typically using transparent rubber material. Twin injection moulding (or “co-moulding”) may also be used, to allow for the use of rubber of different hardness grades for different parts of the mask. For example, the moulded rubber may be of greater hardness grade at the visor region and of lower hardness grade for the eye sealing elements. Twin injection moulding also allows the use of more than one material for the construction of the mask, for example transparent silicone for the visor region and polyurethane for the remainder of the mask. If more than two grades of rubber or more than two different materials are required, multiple stage injection moulding may be used.

There may be a single arm extending from either side of the face-covering element of the mask, as shown in FIGS. 2 and 3. Alternatively, there may be two arms extending from each side of the face covering element, as shown in FIGS. 4 and 5.

FIG. 6 shows a visor cover (40) which may be held in place over the visor region, either on the side of the mask adjacent to a wearer's face or on the side of the mask away from the wearer's face. The visor cover may be, for example, made of transparent coloured material or may carry advertising and/or sponsorship logos. It may be of rigid or semi-rigid material, for example polycarbonate.

The skilled person will understand that materials other than those mentioned above may be suitable for the construction of the mask. For example, the visor region may be constructed from hardened plastics material (e.g. polycarbonate) or glass, such that the mask is suitable for use in diving. In addition, the visor region may be incorporated into other products intended for protection of the eyes, for example a gas respirator mask.

### EXAMPLE 2

FIG. 7 shows an alternative embodiment of a device according to the invention, formed by a transparent elastic material (45). The device comprises a face-covering element (50) and arms (55) which define cut-outs (60) having edges (65). Mouldings on the inner, that is, face-facing, surface of the device define eye-covering regions (70). The device has an upper edge (75) and a lower edge (80). Advertising and/or sponsorship logos may appear on the material forming the arms (55) or the face-covering element (50) of the device.

FIG. 8 shows a cross-sectional view of the sheet forming the device according to the invention. The material forming the device is moulded to form L-shaped protrusions, or eye-sealing elements (85) on the inner or face-facing surface of the device, which define each eye-covering region (70). The thickness (shown between the arrows X-X) of the material between the eye-covering regions (70) is greater than the thickness (shown between the arrows Y-Y) of the material at the edges (60) of the cut-outs formed by the arms. This variation in thickness serves to reduce the flexibility and elasticity of the device in the eye-covering regions.

FIG. 9 shows a cross section along the line P-P of an alternative embodiment of a device according to the invention. The thickness (shown between the arrows V-V) of the transparent elastic material between the eye-covering regions (70) is greater than the thickness (shown between the arrows W-W) of the material at the edges (65) of the cut-outs formed by the arms. The material forming the device is also moulded to form L-shaped protrusions, or eye-sealing elements (85) on the inner surface of the device, in which are formed lips (90) which define a groove (95) in the eye-sealing element in which a replaceable transparent rigid sheet may be placed, to cover the eye-covering region defined by the eye-sealing element. Such rigid sheets may comprise a plastics material which may be colourless, may be tinted with transparent colour or may be patterned. The rigid sheets, when held in place by the lips (90) in each eye-sealing element (85), may also serve to further reduce the flexibility of the device in the eye-covering region.

In the embodiments shown in FIGS. 8 and 9, the portions of the L-shaped protrusions which contact the users face are on the inside of the eye-covering region. In an alternative embodiment those portions may extend outwardly (i.e. so the “L” apparent in those figures is orientated in the opposite direction) so as to provide greater user comfort.

FIGS. 10 and 11 show a front and side view of a device according to the invention in use, positioned on the wearer's head. The upper edge (75) is positioned across the forehead and the lower edge (80) is positioned across the wearer's nose and cheeks. The contact made with the wearer's head by the device at the upper (75) and lower (80) edges and at the edges (65) of the cut-outs (60) of the device, as the device is stretched to extend around the wearer's head, creates a seal such that water does not make contact with the wearer's eyes. The eye-sealing elements (85) formed on the inner surface of the device make contact with the wearer's face around, but not in, each eye socket and create a further seal around each eye. Each eye-sealing element also spaces the material of the inner surface of each eye-covering region from the eye. The cut-outs (60) allow the device to be positioned on the wearer's head without covering the ears. Arms (55) can be seen in FIG. 11 to extend around the wearer's head to meet at the rear.

In the embodiment of the invention illustrated in FIG. 12, a rear view of a device according to the invention in use, positioned on the wearer's head, the arms (55) can be seen to be continuous with one another. Alternatively, the arms (55) may have ends which can be releasably fastened together in use with fastening means. FIG. 13 shows a rear view of a device according to the invention positioned on the wearer's head, with ends (95, 100) of each pair of arms (55) separated from one another and folded back to reveal fastening means (105), for example, a hook and loop fastening material such as Velcro@. The ends of each pair of arms may alternatively be fixed together using a buckle device or a clip device.

In an alternative embodiment of the device, the arms of the device are not of unitary construction with the face-covering element of the device. A plan view of a device according to this embodiment of the invention is shown in FIG. 14. The face-covering element (50) of the device is formed by a transparent elastic material (45) which has an upper edge (75) and a lower edge (80) and is shaped at the short edges of the material to form shaped side edges (110) to the device. Arms (115) are formed from a material different to the transparent elastic material and are fixed to the sheet at the short edges (120) above and below each shaped side edge (110). The material forming the arms may be an elastic material, for example, a Lycra®-containing material.

### EXAMPLE 3

A further alternative device according to the invention is now described, in which at least two sheets of material may be bonded together to form a mask. FIG. 15A shows a transparent first elastic sheet (125) which may be, for example, a transparent silicone rubber material. The sheet comprises a main body portion (130) and arms (135) which define cut-outs (140), having edges (145).

FIG. 15B shows a second, typically non-transparent, elastic sheet (150) which may form a component part of a device according to the invention. The second sheet may, for example, be a silicone rubber material as used for swimming caps. Similarly to the first sheet, the second sheet comprises a main body portion (155) and arms (160) which define cut-outs (165), having edges (170). An aperture (175) is formed in the second sheet, comprising two eye spaces (180, 185) which define a moulded nose region (190), which covers the wearer's nose when the device is in use.

FIG. 16 shows the moulded shape of the device in the nose region. The thickness (shown between arrows S-S) of the sheet at the moulded nose region (190) is greater than the thickness (shown between the arrows T-T) of the sheet at the edges (170) of the cut-outs formed by the arms.

FIG. 17 shows how the first (125) and second (150) sheets may be layered together such that the cut-outs of each sheet (140, 165) are aligned. The sheets may be fixed together using, for example, silicone glue, or another suitable adhesive, or other means for joining the sheets. FIG. 18 shows a plan view of a device constructed in this way. The first (125) and second (150) sheets are layered together. The alignment of the cut-outs of each sheet results in cut-outs (195) formed in the assembled device, the cut-outs having edges (200). The alignment of the arms of each sheet creates layered arms (205). The device resulting from the layering of the first and second sheets is a generally non-transparent device with a transparent aperture (175), through which the wearer is able to see when the device is in use, comprising two eye spaces (180, 185) which define a moulded nose region (190). The device has an upper edge (210) and a lower edge (215). Advertising and/or sponsorship logos may appear on the material forming the arms (205) or the main body (155) of the device. Such logos may, for example, be printed onto the second sheet.

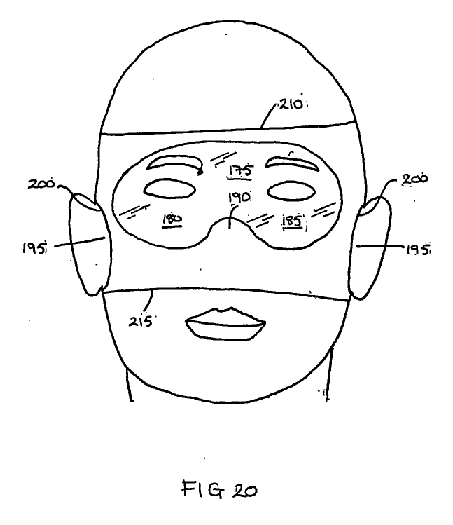

FIG. 19 shows the moulded shape of the nose region of the device illustrated in FIG. 18. The first (125) and second (150) sheets are layered together. The thickness (shown between arrows U-U) of the second sheet (150) at the moulded nose region (190) is greater than the thickness (shown between arrows V-V) of the sheet at the edges (200) of the cut-outs formed by the arms. The first sheet (125) is layered on the inner surface of the second sheet (150). The increased thickness of the second sheet around the nose mould has the result that the material forming the nose mould stretches less than the material forming the main body of the sheet under any given tension. This, coupled with the moulded shape of this part of the device, has the advantageous effect that the device can be positioned on the wearer's head without the uncomfortable deformation of the wearer's nose. The thickness of the first sheet may also be varied in a similar manner, instead of or in addition to any variation in thickness of the second sheet.

Means may be provided, for example in the form of one or more apertures in the nose region, to allow the wearer to inhale or exhale through the nose. In one embodiment the means may provide for exhalation only, for example by inclusion of a suitable valve mechanism.

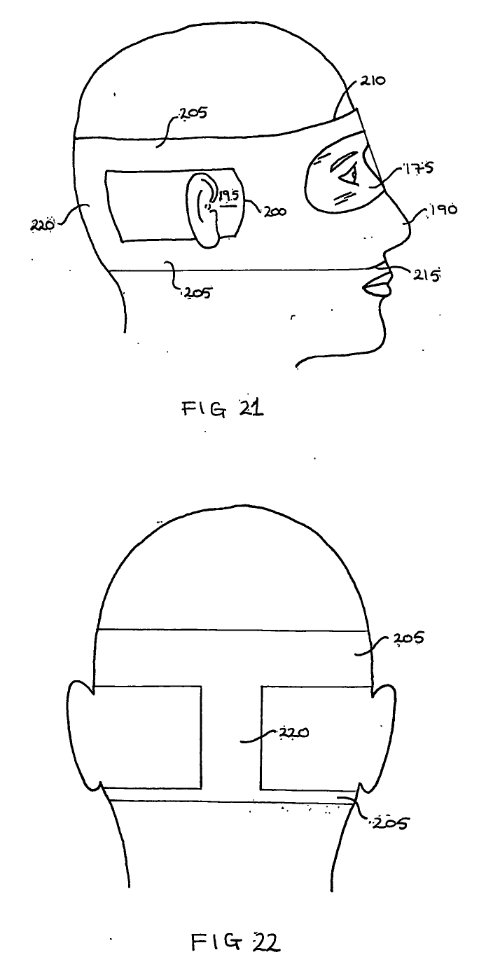

FIGS. 20 and 21 show a front and side view of a device according to the invention in use, positioned on the wearer's head. The upper edge (210) of the device is positioned across the forehead and the lower edge (215) is positioned across the face between the wearer's nose and mouth. The moulded nose region (190) sits over the wearer's nose. The contact made with the wearer's head by the device at the upper (210) and lower (215) edges and at the edges (200) of the cut-outs (195) of the device, as the device is stretched to extend around the wearer's head, creates a seal such that water does not make contact with the wearer's eyes. The cut-outs (195) allow the device to be positioned on the wearer's head without covering the ears. The aperture (175) in the typically non-transparent second sheet allows the wearer to see through the transparent first sheet. Arms (205) can be seen in FIG. 21 to stretch around the wearer's head to meet at a rear band (220). In the embodiment of the invention illustrated in FIG. 22, the arms (205) can be seen to be continuous with the rear band (220).

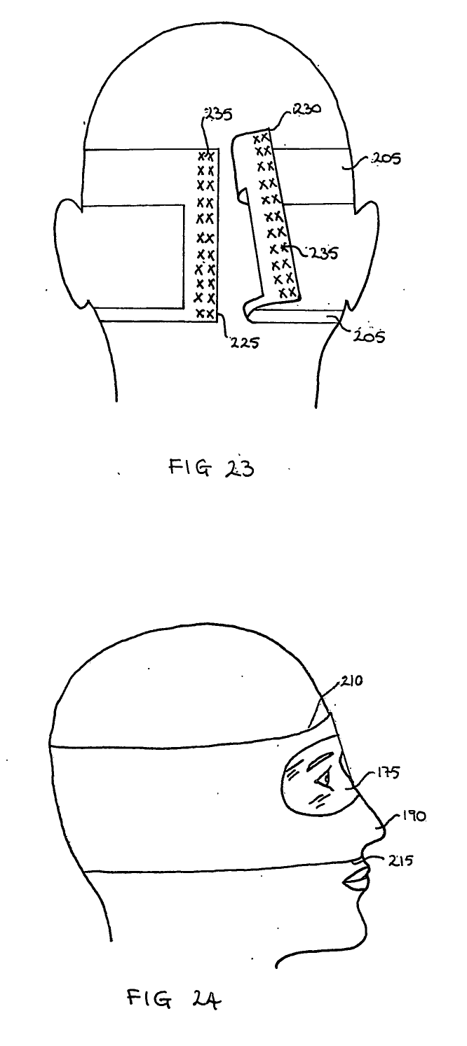

Alternatively, the arms (205) may have ends which can be releasably fastened together in use with fastening means. FIG. 23 shows a rear view of a device according to the invention positioned on a wearer's head, with ends (225, 230) of the arms (205) dividing the rear band. One end (230) is separated from the other (225) and folded back to reveal fastening means (235), for example, a hook and loop fastening material such as Velcro®.

In an alternative embodiment of the device, the device is formed as described above but does not comprise arms. FIG. 24 shows a side view of such a device in use, positioned on the wearer's head. The upper edge (210) of the device is positioned across the forehead and the lower edge (215) is positioned across the wearer's face between the wearer's nose and mouth. The moulded nose region (190) sits over the wearer's nose. The contact made with the wearer's head by the device at the upper (210) and lower (215) edges of the device and by the main body of the device, as the device is stretched to extend around the wearer's head, creates a seal such that water does not make contact with the wearer's eyes. The aperture (175) in the typically non-transparent second sheet allows the wearer to see through the transparent first sheet.

The device may, in an alternative embodiment, be formed from a single transparent sheet.

The skilled person will understand that elements of the embodiments described above may be combined to form a device according to the invention. For example, an injection moulded device as shown in FIGS. 1-5 may be formed lacking arms, as illustrated in FIG. 24, such that the device, in use, covers the ears.

1. Swimming goggles comprising a face-covering element which comprises a visor region formed by a first layer of transparent elastic material, eye sealing elements adapted to sealingly engage with a wearer's face and fastening means which in use hold said goggles in place, wherein in use said first layer is elastically deformable with said eye sealing elements to engage said wearer's face.

2. Swimming goggles according to claim 1, wherein a molded nose region is provided to fit around said wearer's nose.

3. Swimming goggles according to claim 1, wherein said face-covering element is of unitary construction.

4. Swimming goggles according to claim 1, wherein said visor region comprises a single lens region.

5. Swimming goggles according to claim 1, wherein said visor region comprises two lens regions.

6. Swimming goggles according to claim 1, wherein said fastening means comprise a band which, in use, encircles said wearer's head.

7. Swimming goggles according to claim 1, wherein said fastening means comprise arms which, in use, are releasably fastened together.

8. Swimming goggles according to claim 1, wherein the device is formed of a plastics material.

9. Swimming goggles according to claim 1, wherein the visor region is treated with an anti-fog coating.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)
